(19) United States
(12) Patent Application Publication
(10) Pub. No.: US 2023/0321354 A1
(43) Pub. Date: Oct. 12, 2023

(54) AUTOINJECTOR FOR ADMINISTRATION OF MEDICATIONS

(71) Applicant: Emergent Product Development Gaithersburg Inc., Gaithersburg, MD (US)

(72) Inventors: Gaëtan PAINCHAUD, Francheville (FR); Girum YEMANE-TEKESTE, Gaithersburg, MD (US); Marc TODESCO, Tignieu-Jameyzieu (FR)

(21) Appl. No.: 18/043,996

(22) PCT Filed: Sep. 7, 2021
(86) PCT No.: PCT/US2021/049210

§ 371 Date: Mar. 3, 2023

Related U.S. Application Data
(60) Provisional application No. 63/075,718, filed on Sep. 8, 2020.

(51) Int. Cl. A61M 5/24; A61M 5/20; A61M 5/32

(52) U.S. Cl. CPC A61M 5/2448; A61M 5/2066; A61M 5/3287

(57) ABSTRACT

An autoinjector for administering medications. The autoinjector includes an injector unit that includes a cartridge housing, a first carpule to contain a medication for administration and a second carpule to contain a second medication. The carpules are positioned within the interior area of the cartridge housing. The autoinjector further includes an activator unit having an activation housing, a first and second activation engine positioned within an interior area of the activation housing. Each activation engine includes a spring and a piston, such that the spring and piston are co-axial with the carpules. The autoinjector further includes a retainer to retain the springs, a safety disk positioned at a proximal end of the activation housing, and a safety element to engage the safety disk and maintain the activator unit in a storage setting.

### STATEMENT REGARDING FEDERALLY SPONSORED RESEARCH OR DEVELOPMENT

This invention was made with government support under grant number MCDC W15QKN-16-9-1002 awarded by Department of Defense. The government has certain rights in the invention.

### FIELD

Embodiments described herein generally relate to an injection device. Specifically, embodiments described herein relate to an autoinjector for automatically administering at least two medications.

### BACKGROUND

Activators are components of medical hypodermic syringes that can be operated automatically, and are generally for a single-use. Such a syringe, commonly referred to as an “autoinjector,” is placed with its distal end on an injection site at a region of a body of a subject and is then triggered by a manual action, often taken at an opposite, proximal end of the autoinjector. The terms “proximal” and “distal” refer to the perspective of a person using the autoinjector to administer a medication to a patient. A needle extends from the distal end of the autoinjector.

Autoinjectors are often used to quickly administer life-saving drugs under stressful circumstances, for example, during anaphylaxis resulting from an allergic reaction or after exposure to toxic materials or chemicals.

### SUMMARY

In accordance with embodiments described herein, an autoinjector device is disclosed that includes an injector unit having a cartridge housing, a first carpule to contain first medication for administration, the first carpule being positioned within an interior area of the cartridge housing, and a second carpule to contain second medication, e.g., for simultaneous or concurrent administration with the first medication, the second carpule being positioned within the interior area of the cartridge housing. In some embodiments, the autoinjector further includes an activator unit that includes an activation housing, a first activation engine positioned within an interior area of the activation housing, the first activation engine including a first spring and a first piston, such that the first spring and the first piston are co-axial with the first carpule, and a second activation engine positioned within the interior area of the activation housing, the second activation engine including a second spring and a second piston, such that the second spring and the second piston are co-axial with the second carpule. In some embodiments, the autoinjector includes a retainer to retain the first spring and the second spring, a safety disk positioned at a proximal end of the activation housing, and a first safety element to engage the safety disk and maintain the activator unit in a storage setting.

In accordance with embodiments described herein, an autoinjector device is disclosed that includes an injector unit having a cartridge housing, a first carpule to contain first medication for administration, the first carpule being positioned within an interior area of the cartridge housing, and a second carpule to contain second medication, e.g., for simultaneous or concurrent administration with the first medication, the second carpule being positioned within the interior area of the cartridge housing. A needle assembly housing is secured to the cartridge housing, a first needle assembly is secured to the first carpule and positioned within an interior area of the needle assembly housing, and includes a first needle guide and a first cannula secured to the first needle guide, and a second needle assembly is secured to the second carpule and positioned within the interior area of the needle assembly housing, and includes a second needle guide and a second cannula secured to the second needle guide. In some embodiments, the autoinjector further includes an activator unit that includes an activation housing, a first activation engine positioned within an interior area of the activation housing, a second activation engine positioned within the interior area of the activation housing, a safety disk positioned at a proximal end of the activation housing, and a first safety element to engage the safety disk and maintain the activator unit in a storage setting.

In any of the various embodiments described herein, an autoinjector may further include a first piston rod connected to a first piston, the first piston rod having an intermediate protrusion, wherein a portion of the first piston rod is positioned within an interior area of a first spring such that a distal end of the first spring abuts a proximal surface of the intermediate protrusion and a distal surface of the intermediate protrusion abuts a distal end of a retainer. In some embodiments, a second piston rod may be connected to a second piston, the second piston rod may have an intermediate protrusion, wherein a portion of the second piston rod is positioned within an interior area of a second spring such that a distal end of the second spring abuts a proximal surface of the intermediate protrusion and a distal surface of the intermediate protrusion abuts a distal end of the retainer.

In any of the various embodiments described herein, an autoinjector may further include a second safety element to engage a safety disk and maintain an activator unit in a storage setting, the second safely element extending parallel to a first safety element.

In any of the various embodiments described herein, an injector unit may further include a needle assembly housing connected to a cartridge housing such that a proximal portion of the needle assembly housing engages a distal portion of the cartridge housing, a first needle assembly to connect to a first carpule, the first needle assembly being positioned within an interior area of the needle assembly housing in a storage setting, and a second needle assembly to connect to a second carpule, the second needle assembly being positioned within the interior area of the needle assembly housing in the storage setting. In some embodiments, the injector unit further includes a proximal sealing member that extends around a proximal end of the needle assembly housing, a first distal sealing member positioned at a first distal opening of the needle assembly housing, and a second distal sealing member positioned at a second distal opening of the needle assembly housing. In some embodiments, the first carpule may extend through the proximal sealing member and the cannula of the first needle assembly may be configured to extend through the first distal sealing member. In some embodiments, the second carpule may extend through the proximal sealing member and the cannula of the second needle assembly is configured to extend through the second distal sealing member. In some embodiments, a distal end of the cartridge housing may abut the proximal sealing member.

In any of the various embodiments described herein, a proximal portion of a cartridge housing may engage a distal portion of an activation housing. In some embodiments, the cartridge housing may engage the activation housing via a snap-fit connection.

In any of the various embodiments described herein, a first safety element may extend along an outer surface of a retainer. In some embodiments, the first safety element may extend along an outer surface of a first side of the retainer and a second safety element may extend along the outer surface of a second side of the retainer.

In any of the various embodiments described herein, a first safety element may be planar and include a base, a first arm and a second arm extending from the base with the first and second arms being separated from one another by a space, wherein each of the first arm and the second arm comprises a flange configured to engage a safety disk. In some embodiments, an autoinjector may further include a safety cap removably securable to a proximal end of an outer housing, wherein the safety cap comprises a safety pin positioned in the space between the first arm and the second arm of the first safety element when the safety cap is secured to the proximal end of the outer housing.

In any of the various embodiments described herein, an autoinjector may further include an engine rod, the engine rod having a proximal end and a proximal surface at the proximal end that abuts a distal surface of a safety disk, wherein a proximal end of a first spring abuts a distal surface of a spring support at the proximal end of the engine rod, and wherein a proximal end of a second spring abuts the distal surface. In some embodiments, the engine rod may extend through a bore of a retainer, such that the retainer is movable along the engine rod.

In any of the various embodiments described herein, a needle assembly housing may be secured to a cartridge housing via a snap-fit connection.

In any of the various embodiments described herein, a first needle assembly may be connected to a distal end of a first carpule via a needle clip, wherein the first needle assembly is movable relative to the needle clip.

### BRIEF DESCRIPTION OF THE DRAWINGS

The accompanying drawings, which are incorporated herein and form a part of the specification, illustrate the present disclosure and, together with the description, further serve to explain the principles thereof and to enable a person skilled in the pertinent art to make and use the same.

FIG. 1 shows a perspective view of an autoinjector according to an embodiment.

FIG. 2 shows an exploded view of the autoinjector of FIG. 1.

FIG. 3 shows an exploded view of the autoinjector of FIG. 1 having an alternate end cap.

FIG. 4A shows a perspective view of a carpule of the autoinjector of FIG. 1.

FIG. 4B shows a perspective view of a carpule of an autoinjector according to an embodiment.

FIG. 5 shows a perspective view of a needle assembly and a carpule of the autoinjector of FIG. 1.

FIG. 6A and FIG. 6B show sectional views as taken along line A-A of FIG. 5 of a needle guide and a needle clip of the autoinjector of FIG. 1 in different positions.

FIG. 7 shows a top-down view of a needle guide of a needle assembly of the autoinjector of FIG. 1.

FIG. 8 shows a perspective view of a retainer of the autoinjector of FIG. 1.

FIG. 9 shows a perspective view of a portion of a safety element engaged with a safety disk of the autoinjector of FIG. 1.

FIG. 10 shows a sideview of a safety element of the autoinjector of FIG. 1.

FIG. 11A, FIG. 11B, FIG. 11C, FIG. 11D, FIG. 11E, and FIG. 11F show sectional views as taken along line B-B of the autoinjector of FIG. 1 in different stages of operation.

FIG. 12 shows a sectional view of the autoinjector of FIG. 1 as taken along B-B of FIG. 1.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the claims. The terms “distal” and “proximal”, when used in the following description refer to a position of an end, element or feature relative to an injection site on a body of a subject receiving an injection from the device with “distal” referring to a relative position near the injection site of the subject and with “proximal” referring to a relative position away or distant from the injection site of the subject.

In some situations, it may be beneficial for an injection device to administer more than one medication to a patient, and it may be beneficial to store the medications separately from one another prior to administration. However, storing the medications in separate injection devices may be inconvenient, and it may be cumbersome to store and transport multiple injection devices containing various medications. Further, separately using each injection device, such as by sequentially administering the medication in each injection device may cause additional discomfort to the patient due to administration of a series of separate injections. Instead, it may be desirable to include both medications in a single injection device for administration, e.g., for simultaneous or concurrent administration.

Some embodiments described herein relate to an autoinjector that is configured to administer (e.g., simultaneously or concurrently) two or more medications. In order to administer (e.g., simultaneously or concurrently) two medications, the autoinjector may have two carpules for storing medications that are arranged in a side-by-side orientation such that the carpules are parallel to one another. In some embodiments, the autoinjector may further include two safety elements that are configured to maintain the autoinjector in a storage setting to prevent actuation of the autoinjector. The two safety elements may be arranged parallel to one another and each safety element engages a safety disk so as to retain the autoinjector in the storage setting.

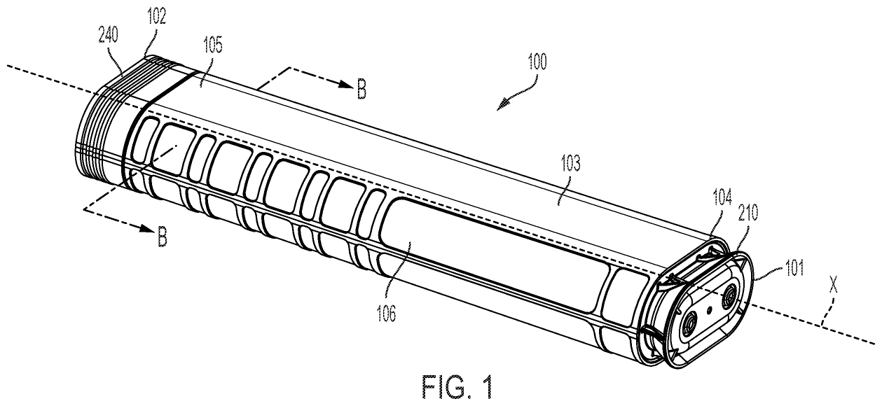

Some embodiments described herein relate to an autoinjector 100 that includes a distal end 101 directed towards a patient when in use to administer the stored medications, and a proximal end 102 opposite distal end 101 and facing away from the patient, as shown in FIG. 1. Autoinjector 100 may be configured for a single use. Autoinjector 100 may be provided with a number of carpules for storing the desired medications in the desired amounts such that the stored doses of the medications may be, for e.g., simultaneously or concurrently administered by the autoinjector 100. Autoinjector 100 may be provided in a storage setting in which springs are compressed, such that when autoinjector 100 is needed to administer a treatment, a user must simply actuate autoinjector 100 to administer the stored medications. As autoinjector 100 provides the force for puncturing chemical protective gear, undergarments, and the skin of a patient to deliver the stored medications, autoinjector 100 can be used without assistance from or supervision by a medical professional and can be self-administered by a patient. Once autoinjector 100 is used to administer the stored medication, autoinjector 100 can be disposed of or recycled.

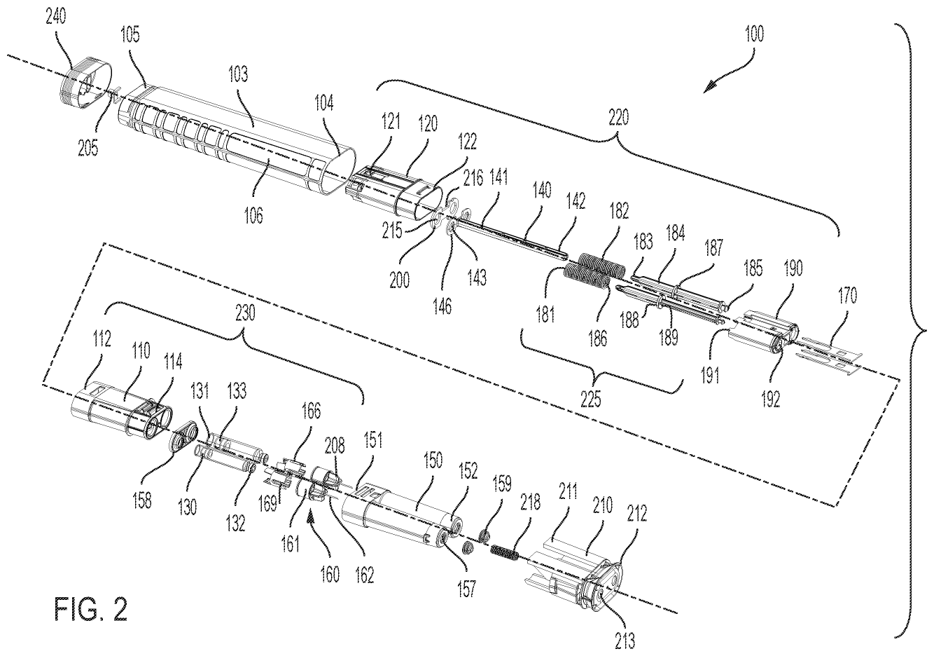

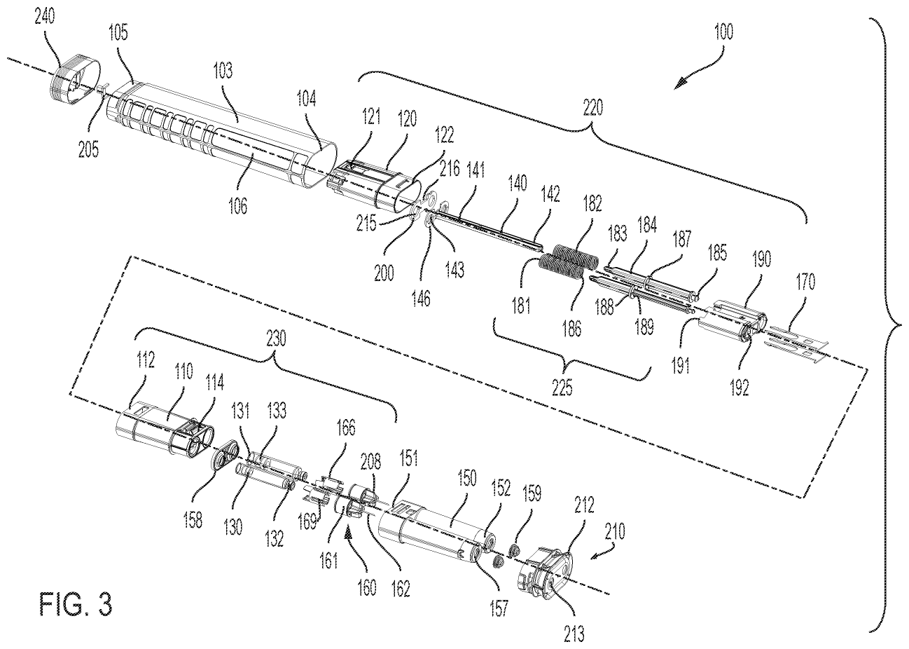

Autoinjector 100 may have an outer housing 103 (see, e.g., FIG. 1, FIG. 2, and FIG. 3). Outer housing 103 may enclose an activator unit 220 and an injector unit 230 of autoinjector 100. Outer housing 103 may be in the form of a tubular sleeve having a distal end 104 opposite a proximal end 105. A longitudinal axis X of the autoinjector 100 extends from the distal end 101 to the proximal end 102 thereof. In some embodiments, outer housing 103 includes a transparent portion 106 configured to allow a user to view the amount of medication stored within the autoinjector 100. In this way, the user may confirm whether autoinjector 100 has already been administered and if the medication stored therein has been fully dispensed.

Outer housing 103 is movable in relation to an activation housing 120 of activator unit 220 so as to cause autoinjector 100 to administer the stored medication. In some embodiments, outer housing 103 may slide relative to activation housing 120 in a direction of the longitudinal axis X of autoinjector 100 from proximal end 102 toward distal end 101, i.e., in a direction toward the patient.

With reference to FIG. 2 and FIG. 3, in some embodiments, autoinjector 100 includes an injector unit 230 having a plurality of carpules 130 for storing medication to be administered and needle assemblies 160 for delivering the stored medication to a patient, and an activator unit 220 including an activation engine 225 for each carpule 130 that includes a spring 182 and a piston 133. Autoinjector 100 may further include a safety element 170 that engages a safety disk 200 to retain autoinjector 100 in a storage setting with springs 182 in a compressed state until autoinjector 100 is ready to be used to administer the stored medication. Upon release of safety element 170 from safety disk 200, activation engines 225 cause administration of medication from each carpule 130.

Autoinjector 100 includes an injector unit 230, as shown in FIG. 2 and FIG. 3. Injector unit 230 may include a cartridge housing 110 that defines an interior area 116 and a proximal end 112 opposite a distal end 114. One or more carpules 130 are positioned within an interior area 116 of cartridge housing 110. Carpules 130 are configured to store a volume of medication for administration to a patient.

Autoinjector 100 may include a single carpule, two carpules, or three or more carpules, depending upon the number of medications desired to be, for e.g., simultaneously or concurrently administered. In embodiments having multiple carpules, each carpule may be identical or may be different. In embodiments having two or more carpules, carpules may have different dimensions and/or volumes so as to store different amounts of medication, and in some embodiments the carpules may differ in cross-sectional shape. For convenience and brevity, the construction of a single carpule will be described, and it is understood that any number of carpules may be included in autoinjector 100 that are constructed in accordance with the following description.

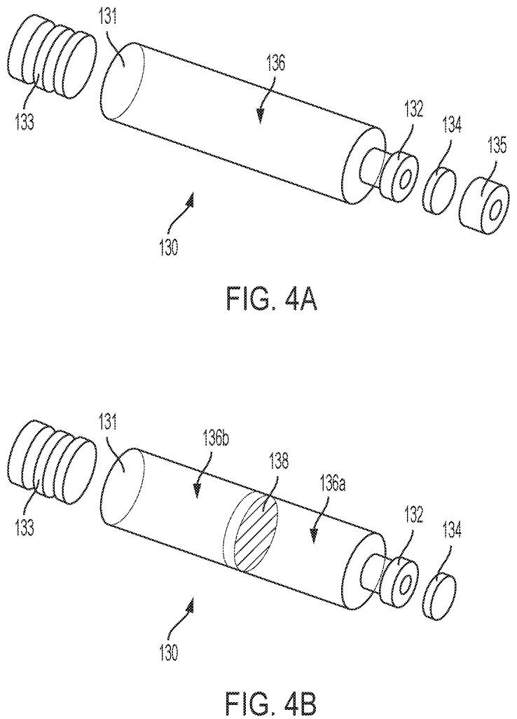

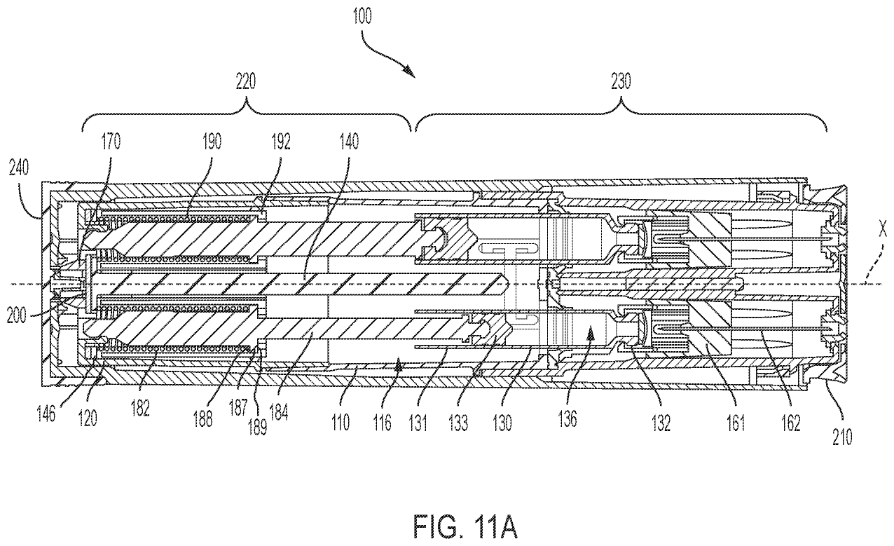

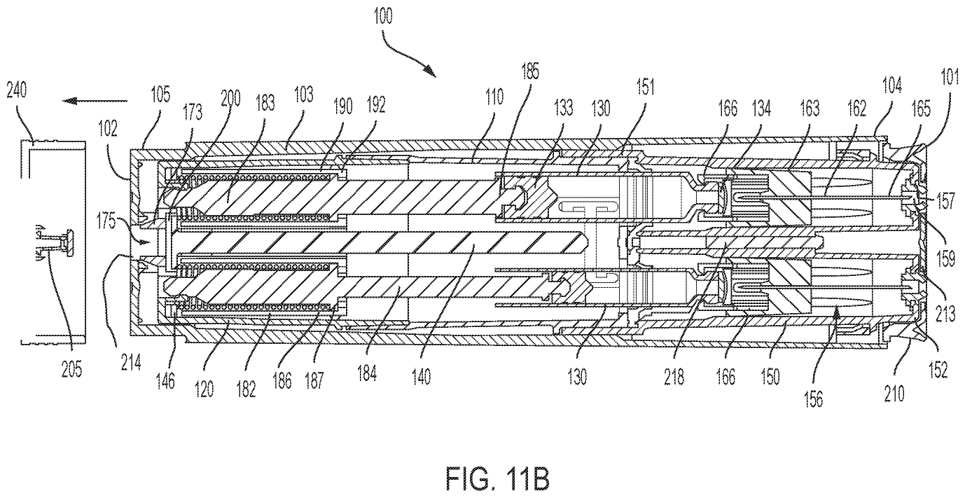

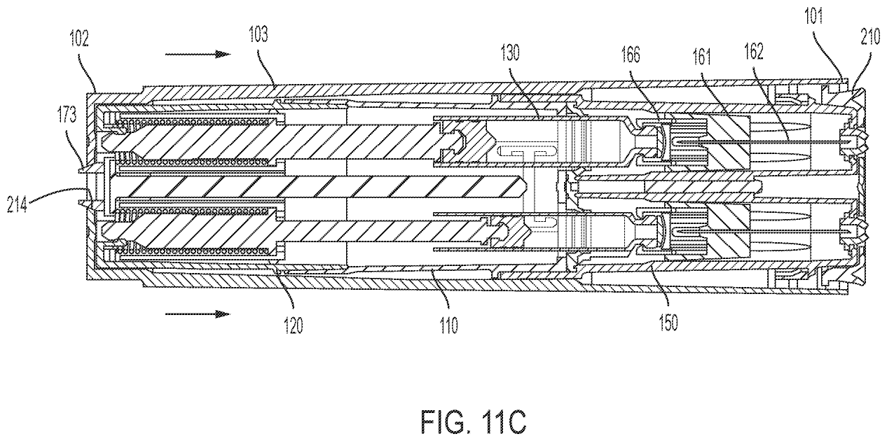

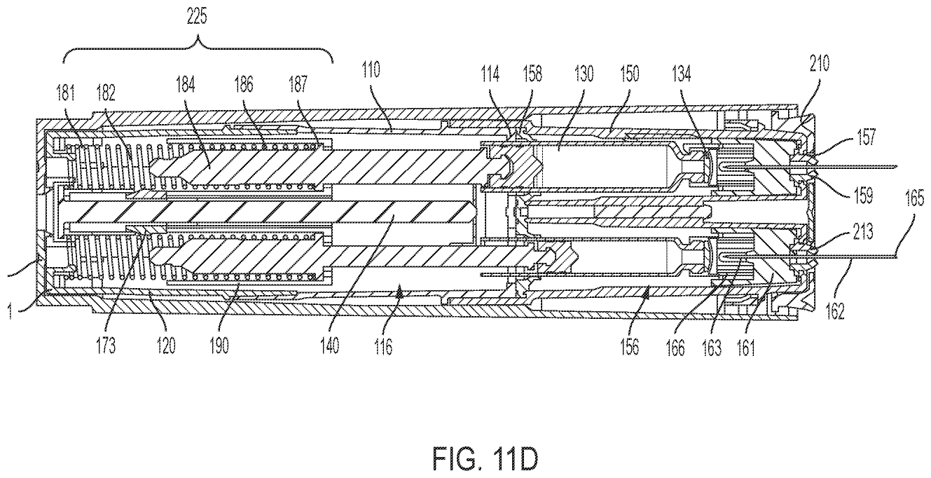

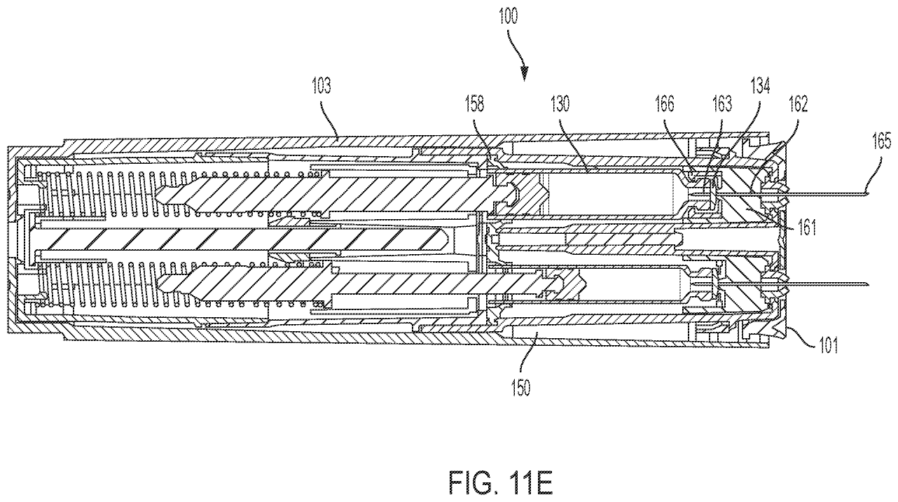

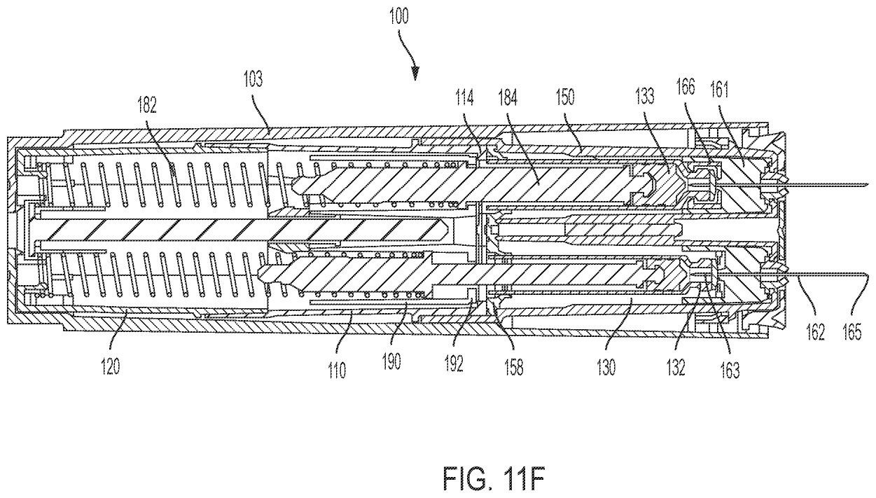

A carpule 130 is configured to store a medication for administration to a patient, as shown for example in FIG. 4A. Carpule 130 is positioned within an interior area 116 of cartridge housing 110 (see, e.g., FIG. 11A). Carpule 130 defines an interior volume 136 for storing a medication. In some embodiments, interior volume 136 of carpule 130 may store about 0.1 mL to about 5 mL, or about 0.2 mL to about 4, or about 0.5 mL to 2 mL. Carpule 130 may have a tubular structure with an open distal end 132 and an open proximal end 131. In some embodiments, carpule 130 may have a cylindrical configuration such that carpule 130 has a circular transverse cross-section, however, carpule 130 may have any of various other shapes and may have an oval, square, rectangular or triangular transverse cross-section in alternate embodiments. Carpule 130 may be composed of a material that is transparent (e.g., glass) such that a medication within carpule 130 is visible from an exterior of carpule 130.

A plunger or piston 133 of an activation engine 225 is positioned in an interior volume 136 of carpule 130 at a proximal end 131 of carpule 130 when autoinjector 100 is in a storage setting. When autoinjector 100 is in use, activation engine 225 is configured to drive piston 133 from proximal end 131 toward distal end 132 of carpule 130 to dispense the medication stored within carpule 130. Piston 133 further serves as a seal to prevent medication from escaping proximal end 131 of carpule 130.

A septum 134 may be disposed at a distal end 132 of carpule 130, as best shown in FIG. 4A. Septum 134 is configured to seal distal end 132 to prevent medication from escaping carpule 130 unintentionally and is further configured to be punctured by a cannula 162 to allow medication to escape carpule 130 via cannula 162. Septum 134 is configured to receive a cannula 162 of a needle assembly 160 therethrough, such that cannula 162 extends through septum 134 and is in fluid communication with the stored medication so as to allow for administration of the stored medication via cannula 162. In some embodiments, a gasket or seal 135 may further be positioned on distal end 132 of carpule 130 to provide additional sealing.

In some embodiments, carpule 130 may be configured to store two or more medications, as shown in FIG. 4B. In this way, autoinjector 100 may be used to deliver various combinations of medications to a patient without requiring a separate carpule and needle assembly for each medication. Carpule 130 of FIG. 4B is substantially the same as carpule 130 of FIG. 4A, and differs only in that one or more additional septum 138 are positioned within the tubular body of carpule 130 at a location between proximal end 131 and distal end 132. Septum 138 within the tubular body of carpule 130 divides the interior volume of carpule 130 into a first volume 136a and a second volume 136b on opposing sides of septum 138. Septum 138 is positioned transversely to a longitudinal axis of carpule 130 so that the first volume 136a is positioned adjacent distal end 132, and the second volume 136b is positioned adjacent proximal end 131 of carpule 130. In this way, a first medication may be stored in first volume 136a of carpule 130 and a second medication may be stored in second volume 136b of carpule 130. It is understood that additional septum 138 may be positioned within carpule 130 to create additional volumes/areas for storing a third or subsequent medication. In this way, a single carpule 130 may contain multiple medications for administration to a patient. As plunger 133 is pushed from proximal end 131 to distal end 132 of carpule 130, the first medication in the first volume 136a is dispensed through distal end 132 (e.g., through cannula 162 extending through septum 134, as explained in more detail below), and septum 138 is pushed towards distal end 132. As septum 138 reaches cannula 162 (e.g., after the first medication is dispensed), cannula 162 may pierce septum 138 to thereby access and dispense the second medication in the second volume 136b.

In some embodiments, autoinjector 100 may include two carpules, such as a first carpule 130 that stores a first medication and a second carpule 130 that stores a second medication. As discussed above, second carpule 130 may be formed in the same manner as first carpule 130. The second medication stored in second carpule 130 may be the same as the first medication, or the second medication may be a different medication. Similar to first carpule 130, second carpule 130 is also positioned within interior area 116 of cartridge housing 110. In the example embodiments shown in the figures, autoinjector 100 includes two carpules 130 (i.e., a first carpule 130 and a second carpule 130).

First and second carpules 130, and any further carpules 130, may be arranged parallel to one another and to longitudinal axis X of autoinjector 100 (see FIG. 2 and FIG. 3). In embodiments having three or more carpules 130, carpules 130 may be arranged parallel to one another in a single plane, or carpules 130 may be arranged in a triangular configuration, or a square configuration, when viewed in a transverse cross-section of autoinjector 100, depending upon the number of carpules 130.

Autoinjector 100 may further include a needle assembly housing 150, as shown, for example, in FIG. 2 and FIG. 3. Needle assembly housing 150 defines an interior area 156 and includes a proximal end 151 opposite a distal end 152. In some embodiments, distal end 152 includes distal openings 157, such as a first distal opening 157 and a second distal opening 157. A distal sealing member 159 may be positioned in each distal opening 157. Distal sealing member 159 helps to prevent dirt and debris from entering the autoinjector through distal openings 157. Proximal end 151 of needle assembly housing 150 engages a distal end 114 of cartridge housing 110. In some embodiments, needle assembly housing 150 may engage cartridge housing 110 via a snap-fit connection, however, in alternate embodiments, needle assembly housing 150 may be secured to cartridge housing 110 via various types of connection, such as by male and female connections, interference fit, among others.

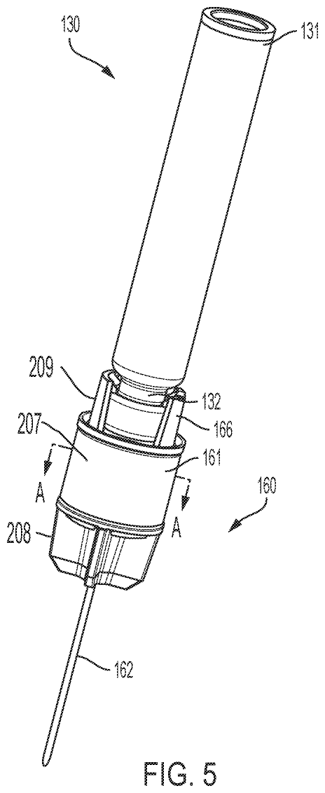

A needle assembly 160 is connected to a carpule 130, as shown in FIG. 5, and needle assembly 160 is positioned within the interior area 156 of needle assembly housing 150 with autoinjector 100 in a storage setting. Needle assembly 160 includes a needle guide 161 and a cannula 162 having a proximal end 163 and a distal end 165. Needle guide 161 supports and surrounds a portion of cannula 162. Needle assembly 160 may further include a needle clip 166 that connects needle guide 161 to distal end 132 of the carpule 130. Needle clip 166 may engage a portion of needle guide 161 and a distal end 132 of carpule 130, such that cannula 162 is coaxial with carpule 130.

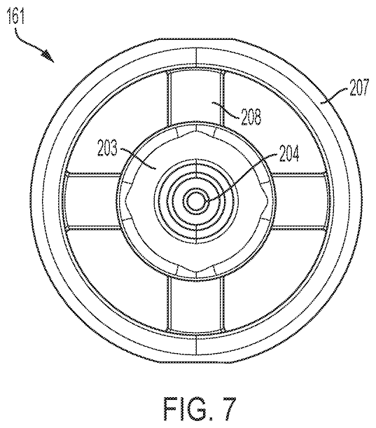

In some embodiments, needle guide 161 comprises a tubular shell 207 and a support 203 having an aperture 204 to receive a cannula 162 therethrough, as shown, for example, in FIG. 7. Cannula 162, also referred to as a “needle,” may be a thin hollow tube through which fluid, such as a medication may flow for delivery to a patient. Support 203 may be positioned centrally within tubular shell 207 and connected to tubular shell 207 by one or more radially extending bars 208, for e.g., four bars 208 as shown in the embodiment of FIG. 7. Cannula 162 extends along a longitudinal axis of needle guide 161 (and parallel to a longitudinal axis X of autoinjector 100). Needle guide 161 is configured to hold cannula 162 in a fixed relationship relative to needle guide 161.

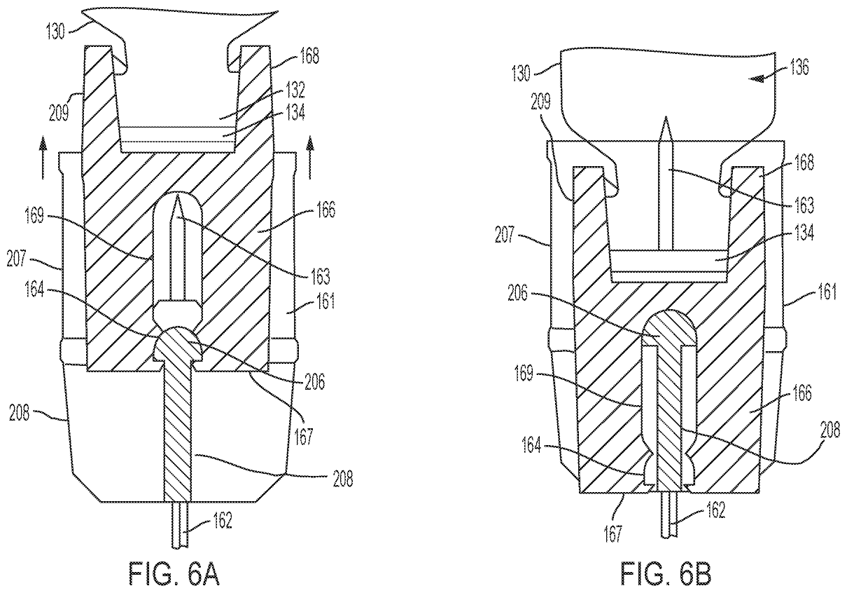

In embodiments of autoinjector 100 having a needle clip 166, as is shown for example in FIG. 6A and FIG. 6B, needle clip 166 may be movably/slidably connected to needle guide 161. Needle clip 166 includes a proximal end 168 secured to distal end 132 of carpule 130, such as about an exterior of distal end 132 of carpule 130, for e.g., by proximal extensions 209, and includes slot(s) 169 proximally extending from a distal end 167 thereof. Needle clip 166 may be fixed in position relative to carpule 130 by proximal extensions 209. Needle clip 166 is received within tubular shell 207 of needle guide 161 and is movably secured to needle guide 161 via engagement of bar(s) 208 of needle guide 161 with slot(s) 169 of needle clip 166. The needle guide 161 and the needle clip 166 each include one of a female connector or a male connector for engaging with a corresponding male connector or female connector of the other in order to secure or lock a relative position of the needle guide 161 and the needle clip 166 components in a storage setting and thereby prevent accidental or premature puncturing of the septum 134 of the carpule 130 by the cannula 162, as further described below. For instance, each slot(s) 169 of needle clip 166 includes a female connector or notch 164 at distal end 167 of needle clip 166 in which a respective male connector or radially-extending proximal end 206 of a bar 208 of needle guide 161 is positioned, to secure or lock the relative positions thereof when autoinjector 100 is in a storage setting. In this position, needle guide 161 supports cannula 162 adjacent to septum 134 of carpule 130 (see FIG. 6A). It should be understood from the foregoing description that other male and female connectors may be utilized to secure or lock the relative positions of the needle guide and the needle clip in a storage setting without departing from the scope hereof, and that a needle clip may include a male connector and a needle guide a corresponding female connector without departing from the scope hereof.

When autoinjector 100 is actuated to administer a medication, carpule 130 is driven towards a distal end 101 of autoinjector 100, and as carpule 130 (and needle clip 166 attached thereto) is driven towards distal end 101, needle guide 161 slides on needle clip 166 toward carpule 130. Specifically, the male connector(s) 206 of bar(s) 208 disengage from the female connector(s) or notch(es) 164, and the bar(s) 208 slide within slot(s) 169 towards proximal end 168 of needle clip 166. As needle guide 161 slides toward carpule 130, cannula 162 fixed thereto also moves towards carpule 130 and may puncture septum 134 of carpule 130 so as to insert proximal end 163 of cannula 162 into interior volume 136 of carpule 130 (see FIG. 6B).

Needle clip 166 may define a slot 169 extending from distal end 167 toward proximal end 168 configured to receive a bar 208 of needle guide 161 such that needle guide 161 may slide relative to needle clip 166 in a longitudinal direction. Distal end 167 of slot 169 may include a notch 164 so as to secure needle guide 161 in a first position with respect to needle clip 166 in a storage setting, and when autoinjector 100 is actuated, the bar(s) 208 of the needle guide 161 slide into slot(s) 169 of needle clip 166 toward proximal end 168 of needle clip 166.

In some embodiments, autoinjector 100 may include a second needle assembly 160 that is constructed in the same manner as first needle assembly 160, and second needle assembly 160 connects to a second carpule 130 in the same manner that first needle assembly 160 connects to first carpule 130, such as by a needle clip 166. Thus, the number of needle assemblies 160 corresponds to the number of carpules 130 in autoinjector 100.

In some embodiments, a proximal sealing member 158 may abut a distal end 114 of cartridge housing 110, as shown in FIG. 2 and FIG. 3. First carpule 130 may extend through proximal sealing member 158. Proximal sealing member 158 helps to prevent dirt or debris from entering cartridge housing 110. Similarly, a second carpule 130 may extend through proximal sealing member 158. When autoinjector 100 is used to administer medication, first carpule 130 may move with respect to proximal sealing member 158 into needle assembly housing 150 as first carpule 130 is driven by activation engine 225 toward a distal end 101 of autoinjector 100.

Autoinjector 100 further includes an activator unit 220, as shown in FIG. 2 and FIG. 3, that is configured to administer the medications stored within each carpule 130. Thus, for example, in embodiments having two carpules 130, autoinjector 100 may be configured to simultaneously or concurrently administer the first and second medications stored within first and second carpules 130.

Activator unit 220 may include an activation housing 120. Activation housing 120 includes a proximal end 121 opposite a distal end 122 and defines an interior area 126. In some embodiments, a proximal end 112 of cartridge housing 110 engages a distal end 122 of activation housing 120. Cartridge housing 110 may engage activation housing 120 via a snap-fit connection. However, in alternate embodiments, the connection may be by male-female connections, interference fit, bayonet connectors, or may be by glue, adhesives, welding, such as ultrasonic welding, or the like.

An activation engine 225 is positioned within interior area 126 of activation housing 120. Activation engine 225 is configured to drive the dispensing of medications from each carpule 130. The number of activation engines 225 may correspond to the number of carpules 130. Thus, in embodiments having two carpules 130, autoinjector 100 may include two activation engines 225. First activation engine 225 is configured to drive administration of medication from first carpule 130, and in embodiments having a second carpule 130, a second activation engine 225 is configured to drive administration of medication from second carpule 130.

Each activation engine 225 may be configured in the same manner as described herein. For simplicity, the configuration of a single activation engine 225 will be described and it is understood that additional activation engines, such as second activation engine and subsequent activation engines, may be constructed in accordance with the following description.

First activation engine 225 may include a first drive element, such as a first spring 182, and a first piston 133. First spring 182 and first piston 133 may be positioned coaxially with first carpule 130. First spring 182 is configured to drive first carpule 130 toward distal end 101 of autoinjector 100, which causes a cannula 162 of a needle assembly 160 connected to first carpule 130 to extend through needle assembly housing 150 of autoinjector 100, and further to drive first piston 133 toward distal end 132 of first carpule 130 so as to drive medication stored in first carpule 130 to flow through its respective cannula 162 for administration to a patient.

In some embodiments, activation engine 225 further includes a first piston rod 184 connected to first piston 133. A distal end 185 of first piston rod 184 may be connected to first piston 133 by any of various fastening methods, such as by glue or adhesives, male-female connections, welding, bonding, or the like. A portion of first piston rod 184 adjacent a proximal end 183 of piston rod 184 is positioned within an interior area of first spring 182.

First piston rod 184 may further include an intermediate protrusion 187 located on first piston rod 184 at a location between proximal end 183 and distal end 185. Intermediate protrusion 187 extends in a transverse direction relative to a longitudinal axis of first piston rod 184 such that a distal end 186 of first spring 182 abuts a proximal surface 188 of intermediate protrusion 187. In some embodiments, intermediate protrusion 187 may extend circumferentially from piston rod 184.

In some embodiments, autoinjector 100 further includes an engine rod 140, as shown in FIG. 2 and FIG. 3. Engine rod 140 includes a proximal end 141 opposite a distal end 142. Engine rod 140 may be substantially linear in configuration and may extend along the longitudinal axis X of autoinjector 100. Engine rod 140 includes a spring support 146 at proximal end 141. A proximal end 181 of first spring 182 abuts a distal surface 143 of the spring support 146. Thus, first spring 182 extends between, and is bounded by, spring support 146 and intermediate protrusion 187.

In embodiments having two activation engines 225, and thus two springs 182, springs 182 may be positioned on opposing sides of engine rod 140 and extend parallel to engine rod 140.

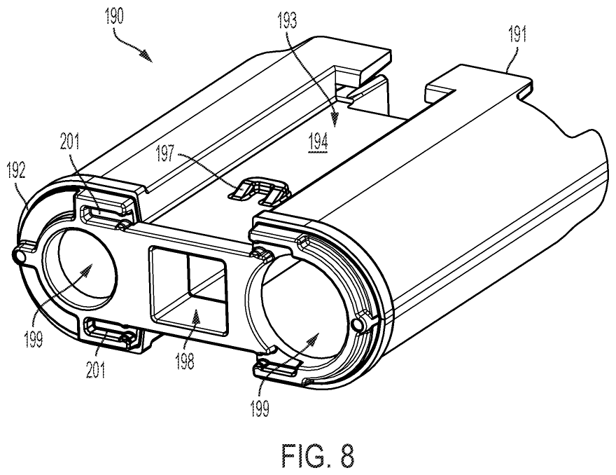

In some embodiments, a retainer 190 as shown in FIG. 8, is configured to retain spring 182 of activation engine 225, and in embodiments having two springs 182 may retain first and second springs 182 of activation engines 225. Retainer 190 includes a proximal end 191 opposite a distal end 192 and may define a chamber 199 for each spring 182. In a storage setting, retainer 190 is positioned within activation housing 120 of activator unit 220, and when autoinjector 100 is used to administer medication, retainer 190 may move toward distal end 101 of autoinjector 100 and partially into cartridge housing 110 as springs 182 extend. Distal end 192 of retainer 190 is secured to a distal surface 189 of intermediate protrusion 187 of piston rod 184. In a storage setting, proximal end 191 of retainer 190 may abut spring support 146 of engine rod 140.

Retainer 190 may further include a central bore 198 extending from proximal end 191 to distal end 192. Engine rod 140 may be received through central bore 198 such that retainer 190 is movably positioned on engine rod 140. Central bore 198 may have a cross-sectional shape corresponding to a cross-sectional shape of engine rod 140. In some embodiments, central bore 198 may have a square or rectangular cross-sectional area so as to receive an engine rod 140 having a square cross-sectional area, such that retainer 190 may not rotate about engine rod 140, ensuring only linear motion of retainer 190 relative to engine rod 140.

Retainer 190 further defines a slot 193 on an outer surface 194 of retainer 190 that extends from proximal end 191 to distal end 192 of retainer 190. Slot 193 is configured to receive and secure a safety element 170. Outer surface 194 of retainer 190 may include a protrusion 197 within slot 193 for facilitating positioning of safety element 170. Further, distal end 192 of retainer 190 may include a recess 201 configured to receive a tab 176 of safety element 170 for further securing and positioning safety element 170 with respect to retainer 190.

When autoinjector 100 is released from a storage setting to administer medication, compressed springs 182 extend and drive piston rods 184 and retainer 190 in a direction toward distal end 101 of autoinjector 100 along engine rod 140. The movement of retainer 190 toward distal end 101 of autoinjector 100 may be limited by distal end 114 of cartridge housing 110.

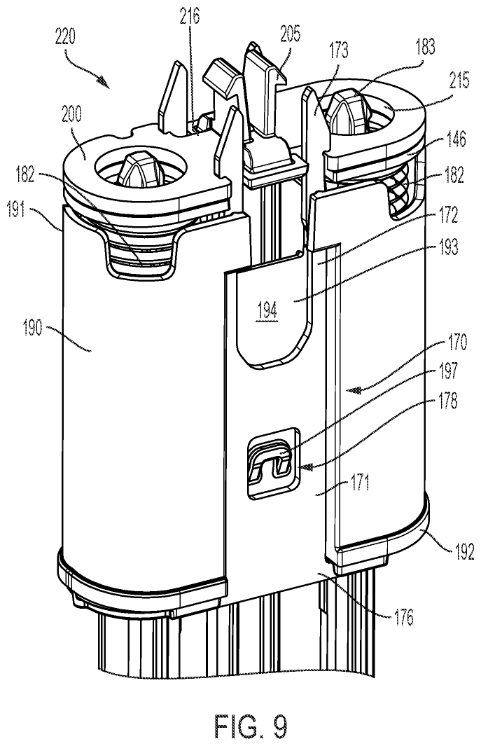

In order to maintain autoinjector 100 so that it is ready for use, with spring 182 in a compressed state, autoinjector 100 may include a safety disk 200 and a safety element 170, as shown in FIG. 9. Safety element 170 engages safety disk 200 so as to retain spring 182 in a compressed state. In some embodiments, safety element 170 and/or safety disk 200 are composed of stainless steel. When safety element 170 is disengaged or released from safety disk 200, springs 182 of activation engines 225 expand from the compressed state, such that activation engines 225 operate simultaneously or concurrently.

Safety disk 200 may have a generally planar configuration. In an embodiment, safety disk 200 may have a flat figure-eight-like shape with apertures 215 configured to receive proximal ends 183 of piston rods 184 when the autoinjector 100 is in its storage setting. In an embodiment, a safety disk 200 may have a flat shape with a waisted area 216 for receiving features of a safety element 170, such as flange(s) 173 discussed below, when the autoinjector 100 is in its storage setting. Safety disk 200 is arranged at a proximal end 121 of activation housing 120, and in an embodiment is fixed thereto, and abuts spring support 146 at proximal end 141 of engine rod 140. Safety disk 200 may be in a fixed position within autoinjector 100. Safety disk 200 is positioned transverse to the longitudinal axis X of autoinjector 100. Safety disk 200 may comprise metal.

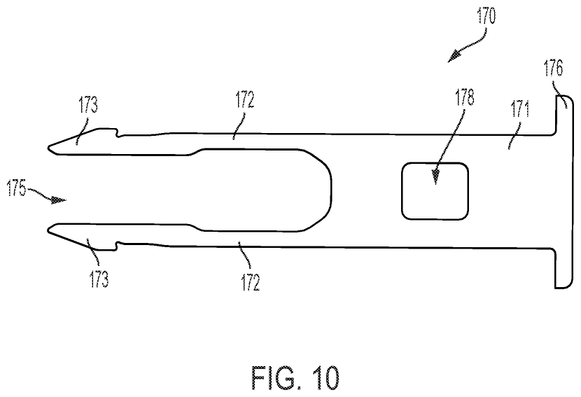

Safety element 170 may be positioned on an outer surface of retainer 190 as shown in FIG. 9. In some embodiments, safety element 170 includes a base 171 and a pair of arms 172 extending from base 171 and are separated by a space 175, as shown in FIG. 10. First safety element 170 may have a generally planar configuration. Further, arms 172 may be arranged generally parallel to one another. An end of each arm 172 opposite base 171 may include a flange 173 for engaging safety disk 200. Flanges 173 may extend outwardly from each arm 172 in a transverse direction, such that flanges 173 on each arm 172 extend in opposing, outward directions. In some embodiments, safety element 170 may comprise metal, such as stainless steel. Base 171 of safety element 170 may include tabs 176 outwardly extending from base 171 so as to engage a recess 201 (see FIGS. 8 and 10) on the distal end 192 of retainer 190 so as to secure safety element 170 to retainer 190. Further, base 171 of safety element 170 may define an opening 178 for receiving a protrusion 197 of retainer 190 to further help to position safety element 170 on an outer surface 194 of retainer 190.

In some embodiments, safety element 170 is positioned on outer surface 194 of retainer 190 and is configured to engage a safety disk 200, as best shown in FIG. 9. With safety element 170 engaged with safety disk 200, retainer 900 retains springs 182 in a compressed state. With safety element 170 positioned on outer surface 194 of retainer 190, arms 172 and specifically flanges 173 of each arm 172 engage a proximal surface of safety disk 200. Safety element 170 extends along outer surface 194 of retainer 190 in a direction of the longitudinal axis X of autoinjector 100. Safety element 170 may be positioned on a first side of retainer 190. An outer surface 194 of retainer 190 on a first side of retainer 190 may define a slot 193 extending from proximal end 191 to distal end 192 to receive safety element 170.

In some embodiments, autoinjector 100 may include two safety elements 170, such as a first safety element 170 and a second safety element 170. In such embodiments, second safety element 170 may be identical to first safety element 170, and thus may include a base 171, a pair of arms 172 extending from base 171 and separated by a space 175, and a flange 173 on each arm 172 as described above. However, second safety element 170 is arranged on an outer surface 194 of retainer 190 on a second side of retainer 190 opposite the first side on which first safety element 170 is positioned. Thus, retainer 190 may be formed with a slot 193 on a second side for receiving second safety element 170. Second safety element 170 also engages safety disk 200. Second safety element 170 may be arranged so that it is generally parallel to first safety element 170. The use of an additional safety element 170 on opposing side of retainer 190 may help to evenly distribute the spring force, and may help to maintain spring(s) 182 in a compressed state in the event first safety element 170 breaks or malfunctions. In such embodiments, first safety element 170 is arranged in a generally parallel orientation with respect to second safety element 170.

In order to disengage first and second safety elements 170 from safety disk 200, first and second arms 172 of each safety element 170 may be moved in a direction toward one another, i.e., toward the longitudinal axis X of the autoinjector. In this way, flanges 173 of arms 172 disengage from safety disk 200, and springs 182 drive retainer 190 and safety elements 170 carried thereon towards distal end 101 of autoinjector 100.

In some embodiments, autoinjector 100 further includes a safety cap 240, as shown in FIG. 1, FIG. 2, and FIG. 3. Safety cap 240 is removably securable to a proximal end 105 of outer housing 103. When safety cap 240 is secured to outer housing 103, autoinjector 100 is retained in a storage setting and cannot be used to administer medication. This helps to prevent autoinjector 100 from accidental or inadvertent operation. In order to utilize autoinjector 100, safety cap 240 must first be removed from the proximal end of the autoinjector 100.

Safety cap 240 may be removably securable to outer housing 103 by an interference fit, snap fit, or the like. In order to prevent use of autoinjector 100 when safety cap 240 is secured to outer housing 103, safety cap 240 may include a safety pin 205. When safety cap 240 is secured to outer housing 103, safety pin 205 is positioned between the pair of arms 172 of safety element 170 so as to maintain engagement of safety element 170 with safety disk 200 (see FIG. 11A). Safety pin 205 may be configured to be inserted in space 175 between arms 172 of a safety element 170 maintain arms 172 spaced from one another. In some embodiments, safety pin 205 may exert a force on arms 172 so as to outwardly bias arms 172 apart from one another, such that when safety pin 205 is released, arms 172 return to an unbiased or resting state in which the arms 172 move inward and closer together.

With arms 172 of the safety element 170 separated by safety pin 205 of safety cap 240, flanges 173 of each arm 172 engage the proximal surface of the safety disk 200. When safety pin 205 is removed, arms 172 are separated by space 175 and are configured to be moved towards one another in order to disengage flanges 173 from the safety disk 200, when appropriate, allowing activation engines 225 to operate.

In operation, autoinjector 100 is maintained in a storage setting with springs 182 in a compressed state, carpules 130 pre-filled with medication, and cannulas 162 of needle assemblies 160 enclosed within autoinjector 100, as shown in FIG. 11A. To use autoinjector 100 to administer medication, safety cap 240 is removed from autoinjector 100 as shown in FIG. 11B. By removing safety cap 240, safety pin 205 of safety cap 240 is removed from a space 175 between pair of arms 172 of each safety element 170.

With safety cap 240 removed from outer housing 103, outer housing 103 may be actuated to cause administration of medication. A user, such as the patient, may position a distal end 101 of autoinjector 100 in contact with an injections site at a region of a body of the patient at which it is desired to administer the stored medications. To actuate autoinjector 100, the user may slide outer housing 103 in a direction toward distal end 101 of autoinjector 100 (i.e., toward the patient), as shown in FIG. 11C. As outer housing 103 moves toward distal end 101 of autoinjector 100, a proximal end 105 of outer housing 103 contacts safety element 170, and particularly flanges 173 of safety element 170 slide within an aperture 214 in the proximal end 105 of outer housing 103 so as to apply an inward force on arms 172 to move arms 172 toward one another, such that flanges 173 disengage from safety disk 200.

When safety elements 170 are disengaged from safety disk 200, springs 182 of activation engine 225 may extend from a compressed state as shown in FIG. 1 ID. Springs 182, which abut with intermediate protrusions 187, push piston rods 184 which in turn push first and second carpules 130, which in turn push needle assemblies 160 toward distal end 101 of autoinjector 100, such that cannulas 162 of needle assemblies 160 extend through and beyond distal end 152 of needle assembly housing 150. As first and second carpules 130 move towards distal end 101 of autoinjector 100, a proximal end 163 of cannula 162 of needle assembly 160 penetrates septum 134 of each carpule 130, as shown in FIG. 11E. As springs 182 continue to push on piston rods 184, pistons 133 are driven within carpule 130 from proximal end 131 to distal end 132 such that the stored medication is dispensed through cannulas 162, as shown in FIG. 11F.

In some embodiments, autoinjector 100 may further include an end cap 210, as shown in FIG. 2. End cap 210 may include a proximal end 211 opposite a distal end 212. Distal end 212 includes a first opening 213 and a second opening 213 to allow cannulas 162 to extend therethrough for administration of medication to a patient. End cap 210 may serve to enclose distal end 104 of outer housing 103. In some embodiments, end cap 210 may be fixed to distal end 104 of outer housing 103, such as by snap fit, interference fit, glue or adhesives, welding, or the like. In some embodiments, however, end cap 210 may be movable from an extended setting in which it is capable of covering the cannulas 162 of autoinjector 100 (such as after autoinjector 100 is used), and a retracted state in which a distal end of end cap 210 abuts distal end 152 of needle assembly housing 150 (when autoinjector 100 is in a storage setting). In such embodiments, end cap 210 may include a spring 218 connected to needle assembly housing 150 in a direction parallel to a longitudinal axis X of autoinjector 100 that biases end cap 210 in an extended position for covering cannulas 162.

In operation of an autoinjector 100 according to an embodiment, autoinjector 100 may be positioned with end cap 210 in contact with a body of a patient, in a location where it is desired to administer the medication, such as for example, a thigh or buttock. Spring 218 is compressed in a retracted setting with distal end 212 of end cap 210 in close proximity to, or abutting, distal end 152 of needle assembly housing 150. Once autoinjector 100 is actuated to administer medication, cannulas 162 of needle assembly 160 extend through needle assembly housing 150 and end cap 210 to administer medication to the patient. When autoinjector 100 is removed from contact with the body of the patient, spring 218 of end cap 210 is released and extends so that end cap 210 covers distal ends 165 of cannulas 162 which project beyond needle assembly housing 150 after administration of the medications. However, in some embodiments, as shown in FIG. 3, end cap 210 may not fully cover the exposed length of cannula 162 after use of autoinjector 100. End cap 210 may simply enclose distal end 104 of outer housing 103 so as to retain needle assembly housing 150 within outer housing 103, and as a result, distal ends 165 of cannulas 162 of autoinjector 100 remain uncovered after use. In such embodiments, a person who administered autoinjector 100 to a patient, or the patient, may bend the exposed cannulas 162 such that a medic who examines the patient may quickly and easily determine that autoinjector 100 has been used so that the medic is aware of the type and dose of medication administered to the patient if the patient is unconscious or is unattended.

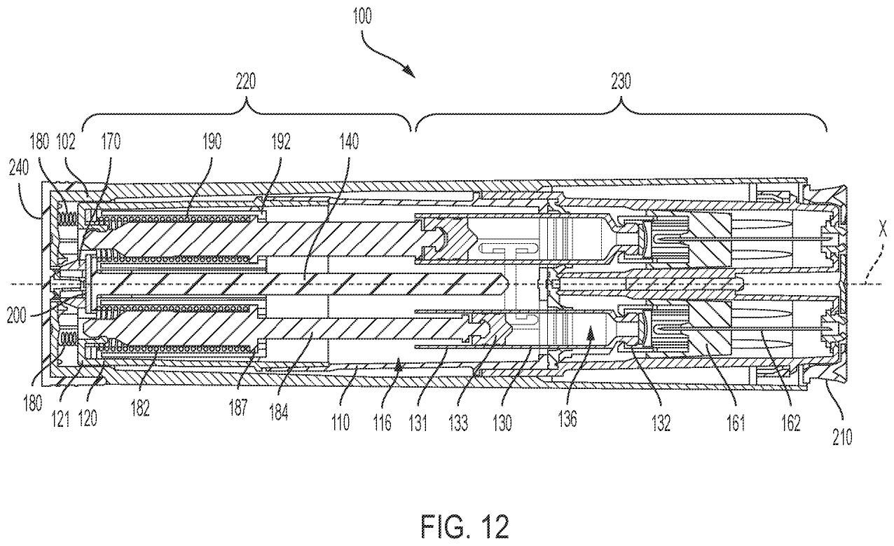

In some embodiments, autoinjector 100 may further include a spring 180 extending between proximal end 102 of outer housing 103 and proximal end 121 of activation housing 120, as shown in FIG. 12. Spring 180 biases activation housing 120 in a spaced relation with outer housing 103 so as to prevent accidental actuation of autoinjector 100 when a user removes safety cap 240 from outer housing 103. Once safety cap 240 is removed, in order to actuate autoinjector 100, a user must apply a sufficient force on outer housing 103 in a longitudinal direction towards distal end 101 of autoinjector 100 to compress spring 180 and move outer housing 103 toward activation housing 120 (and safety elements 170) so that outer housing 103 causes safety element 170 to disengage with safety disk 200, as described above.

It is to be appreciated that the Detailed Description section, and not the Summary and Abstract sections, is intended to be used to interpret the claims. The Summary and Abstract sections may set forth one or more but not all exemplary embodiments of the present disclosure(s) as contemplated by the inventors, and thus, are not intended to limit the present disclosure(s) and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure(s) that others can, by applying knowledge within the skill of the art, readily modify and/or adapt for various applications such specific embodiments, without undue experimentation, and without departing from the general concept of the present disclosure(s). Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance herein.

The breadth and scope of the present disclosure(s) should not be limited by any of the above-described exemplary embodiments, but should be defined only in accordance with the claims and their equivalents.

1. An autoinjector, comprising:
an injector unit, the injector unit comprising:
a cartridge housing;
a first carpule to contain first medication for administration, the first carpule being positioned within an interior area of the cartridge housing;
a second carpule to contain second medication for administration, the second carpule being positioned within the interior area of the cartridge housing;
an activator unit, the activator unit comprising:
an activation housing;
a first activation engine at least partially positioned within an interior area of the activation housing, the first activation engine including a first spring and a first piston, such that the first spring and the first piston are co-axial with the first carpule;
a second activation engine at least partially positioned within the interior area of the activation housing, the second activation engine including a second spring and a second piston, such that the second spring and the second piston are co-axial with the second carpule;
a retainer to retain the first spring and the second spring;
a safety disk positioned at a proximal end of the activation housing; and
a first safety element to engage the safety disk and maintain the activator unit in a storage setting.

2. The autoinjector of claim 1, further comprising a first piston rod connected to the first piston, the first piston rod having an intermediate protrusion, wherein a portion of the first piston rod is positioned within an interior area of the first spring such that a distal end of the first spring abuts a proximal surface of the intermediate protrusion and a distal surface of the intermediate protrusion abuts a distal end of the retainer.

3. The autoinjector of claim 2, further comprising a second piston rod connected to the second piston, the second piston rod having an intermediate protrusion, wherein a portion of the second piston rod is positioned within an interior area of the second spring such that a distal end of the second spring abuts a proximal surface of the intermediate protrusion and a distal surface of the intermediate protrusion abuts a distal end of the retainer.

4. The autoinjector of claim 1, further comprising a second safety element to engage the safety disk and maintain the activator unit in the storage setting, the second safety element extending parallel to the first safety element.

5. The autoinjector of claim 1, the injector unit further comprising:
a needle assembly housing connected to the cartridge housing such that a proximal portion of the needle assembly housing engages a distal portion of the cartridge housing;
a first needle assembly connected to the first carpule, the first needle assembly being positioned within an interior area of the needle assembly housing in the storage setting; and
a second needle assembly connected to the second carpule, the second needle assembly being positioned within the interior area of the needle assembly housing in the storage setting.

6. The autoinjector of claim 1, wherein a proximal end of the cartridge housing engages a distal end of the activation housing.

7. The autoinjector of claim 6, wherein the cartridge housing engages the activation housing via a snap-fit connection.

8. The autoinjector of claim 5, further comprising:
a proximal sealing member that extends around a proximal end of the needle assembly housing;
a first distal sealing member positioned at a first distal opening of the needle assembly housing; and
a second distal sealing member positioned at a second distal opening of the needle assembly housing.

9. The autoinjector of claim 8, wherein the first carpule extends through the proximal sealing member and a cannula of the first needle assembly is configured to extend through the first distal sealing member.

10. The autoinjector of claim 9, wherein the second carpule extends through the proximal sealing member and a cannula of the second needle assembly is configured to extend through the second distal sealing member.

11. The autoinjector of claim 8, wherein a distal end of the cartridge housing abuts the proximal sealing member.

12. The autoinjector of claim 1, wherein the first safety element extends along an outer surface of the retainer.

13. The autoinjector of claim 12, wherein the first safety element extends along the outer surface of a first side of the retainer and a second safety element extends along the outer surface of a second side of the retainer.

14. The autoinjector of claim 1, wherein the first safety element is planar and comprises a base, a first arm and a second arm extending from the base and that are separated from one another by a space, wherein each of the first arm and the second arm comprises a flange configured to engage the safety disk.

15. The autoinjector of claim 14, further comprising a safety cap removably securable to a proximal end of an outer housing of the autoinjector, wherein the safety cap comprises a safety pin positioned in the space between the first arm and the second arm of the first safety element when the safety cap is secured to the proximal end of the outer housing.

16. The autoinjector of claim 1, further comprising an engine rod, the engine rod having a proximal end and a proximal surface at the proximal end that abuts a distal surface of the safety disk, wherein a proximal end of the first spring abuts a distal surface of a spring support at the proximal end of the engine rod, and wherein a proximal end of the second spring abuts the distal surface.

17. The autoinjector of claim 16, wherein the engine rod extends through a bore of the retainer, such that the retainer is movable along the engine rod.

18. An autoinjector, comprising:
an injector unit, the injector unit comprising:
a cartridge housing;
a first carpule to contain first medication for administration, the first carpule being positioned within an interior area of the cartridge housing;
a second carpule to contain second medication for administration, the second carpule being positioned within the interior area of the cartridge housing;
a needle assembly housing secured to the cartridge housing;
a first needle assembly secured to the first carpule and positioned within an interior area of the needle assembly housing, and comprising a first needle guide and a first cannula secured to the first needle guide;
a second needle assembly secured to the second carpule and positioned within the interior area of the needle assembly housing, and comprising a second needle guide and a second cannula secured to the second needle guide;
an activator unit, the activator unit comprising:
an activation housing;
a first activation engine at least partially positioned within an interior area of the activation housing;
a second activation engine at least partially positioned within the interior area of the activation housing;
a safety disk positioned at a proximal end of the activation housing; and
a first safety element to engage the safety disk and maintain the activator unit in a storage setting.

19. The autoinjector of claim 18, wherein the needle assembly housing is secured to the cartridge housing via a snap-fit connection.

20. The autoinjector of claim 18, wherein the first needle assembly is connected to a distal end of the first carpule via a needle clip, wherein the first needle assembly is movable relative to the needle clip.

21. The autoinjector of claim 18, wherein the first carpule comprises a septum that separates an interior volume of the first carpule into a first storage area for storing a first medication and a second storage area for storing a second medication.

22. An autoinjector, comprising:
an injector unit, the injector unit comprising:
a cartridge housing;
a carpule to contain a medication for administration, the carpule being positioned within an interior area of the cartridge housing;
a needle assembly housing secured to the cartridge housing;
a needle assembly positioned within an interior area of the needle assembly housing, the needle assembly comprising a needle guide, a cannula secured to the needle guide, and a needle clip disposed within the needle guide and slidable relative thereto, the needle clip having proximal extensions configured to be secured to a distal end of the carpule;
an activator unit, the activator unit comprising:
an activation housing; and
an activation engine at least partially positioned within an interior area of the activation housing,
wherein when the autoinjector is actuated by the activator unit to administer a medication, the carpule and the needle clip attached thereto are driven towards a distal end of the autoinjector, and as the carpule with the needle clip are driven towards the distal end of the autoinjector, the needle guide slides on the needle clip toward the carpule so as to bring the cannula into fluid communication with the medication contained within the carpule.

23. The autoinjector of claim 22, wherein the needle guide includes longitudinally-extending bars, and the needle clip includes slots that slidably receive the bars therein when the autoinjector is actuated.

24. The autoinjector of claim 23, wherein each slot of the needle clip includes a notch at a distal end of the needle clip in which a radially-extending proximal end of a respective bar of the needle guide is positioned when the autoinjector is in a storage setting.

25. The autoinjector of claim 24, wherein in the storage setting the needle guide supports a proximal end of the cannula adjacent to a septum of the carpule.

26. The autoinjector of claim 22, wherein the needle clip includes one of a male connector or a female connector for engaging with a corresponding female connector or a corresponding male connector of the needle guide, to secure a relative position of the needle clip and the needle guide when the autoinjector is in a storage setting.

27. The autoinjector of claim 26, wherein in the storage setting the needle guide supports a proximal end of the cannula adjacent to a septum of the carpule.
